(19) United States
(12) Patent Application Publication
(10) Pub. No.: US 2013/0274832 A1
(43) Pub. Date: Oct. 17, 2013

(54) ELECTRICAL STIMULATION ENHANCED ULTRASOUND (ESEUS)

(71) Applicant: Michael Keoni Manion, Cronulla (AU)

(72) Inventor: Michael Keoni Manion, Cronulla (AU)

(73) Assignee: Empire Technology Development LLC

(21) Appl. No.: 13/642,481

(22) PCT Filed: Apr. 17, 2012
(86) PCT No.: PCT/US2012/033951

§ 371 Date: Oct. 19, 2012

(51) Int. Cl. A61N 1/36

(52) U.S. Cl. CPC A61N 1/36
USPC 607/72

(57) ABSTRACT

Embodiments provided herein generally relate to electrical stimulation enhanced ultrasound. In some embodiments, a device for ultrasound imaging is provided and includes an ultrasound head and an electrical stimulator. Ultrasound images can be taken before, during, and/or after electrical stimulation to determine if the ultrasound characteristics of a tissue have changed due to the electrical stimulation.

The present disclosure relates generally to the field of ultrasound applications.

### BACKGROUND

Endoscopic ultrasound refers to medical procedures where endoscopy is combined with ultrasound to generate images of internal organs. Endoscopic ultrasound can be used to screen for cancers such as pancreatic cancer, esophageal cancer, and gastric cancer.

### SUMMARY

In some embodiments, a device for ultrasound imaging is provided. The device includes an ultrasound head and an electrical stimulator positioned proximal to the ultrasound head. The electrical stimulator is configured to apply an electrical pulse of less than about 100 V/cm.

In some embodiments, the device can include at least a first electrode configured to contact a tissue near the ultrasound head. The first electrode can include a balloon. The first electrode can include an electrically conductive elastomer. The ultrasound head can be positioned within the electrically conductive elastomer.

In some embodiments, a system for electrical stimulation enhanced ultrasound imaging is provided. The system includes an ultrasound unit, and electrical stimulator, and a controller. The controller is configured to apply an electric pulse of less than about 100 V/cm.

In some embodiments, a method for electric stimulation enhanced ultrasound imaging is provided. The method includes applying an electrical stimulation of less than about 100 V/cm to one or more regions of interest and determining a change in an ultrasound signal caused by applying the electrical stimulation.

In some embodiments, a method for electrical stimulation and ultrasound imaging is provided. The method includes acquiring an ultrasound image from one or more regions of interest, applying a pulse of electrical stimulation to the one or more regions of interest, and acquiring an ultrasound image of the one or more regions of interest during application of the pulse of electrical stimulation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a drawing depicting some embodiments of a device for ultrasound imaging.

FIG. 2 is a drawing depicting some embodiments of a system for electric stimulation enhanced ultrasound imaging.

FIG. 3 is a drawing depicting some embodiments of a method for electric stimulation enhanced ultrasound imaging.

FIG. 4 is a drawing depicting some embodiments of a method for electric stimulation enhanced ultrasound imaging.

FIG. 5 is a drawing depicting some embodiments of a method for electric stimulation enhanced ultrasound imaging.

FIG. 6 is a drawing depicting some embodiments of a method for electric stimulation enhanced ultrasound imaging.

FIGS. 7A and 7B are drawings depicting ultrasound images.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

In some embodiments, a device for ultrasound imaging is provided. The device includes an ultrasound head and an electrical stimulator. The electrical stimulator can be configured to apply an electrical pulse of less than about 100 V/cm.

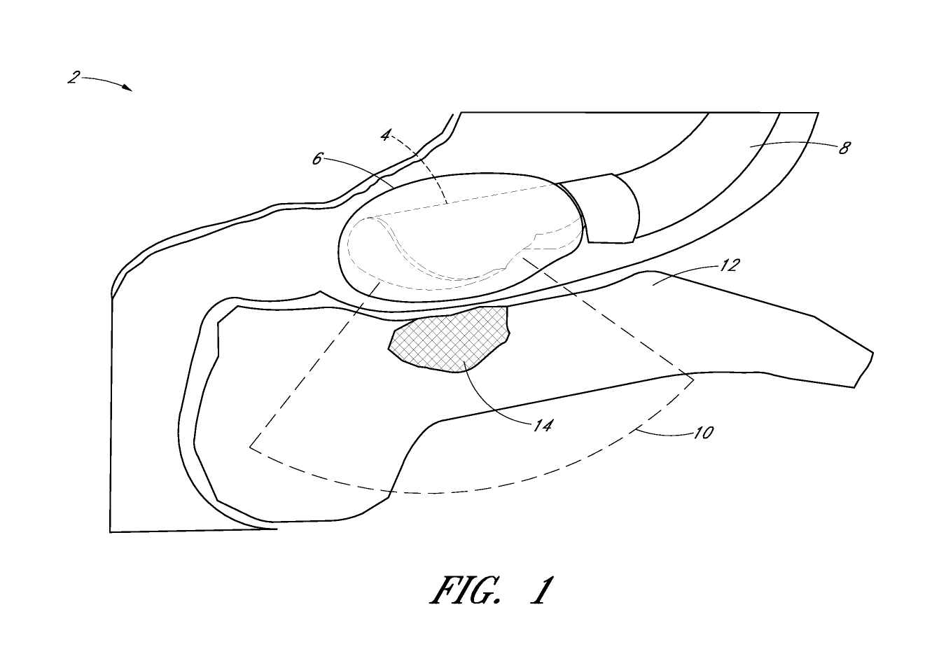

FIG. 1 depicts some embodiments of a device 2 for ultrasound imaging being used within the body of a patient. The device includes an ultrasound head 4 positioned within a balloon electrode 6. Both the ultrasound head 4 and the balloon electrode 6 are positioned at the distal end of an endoscopic probe 8. The ultrasound window and the electrical stimulation window overlap to form an overlap window 10. The device can be used, for example, to examine tissue 12 including a region of interest 14, which can be a tumor.

The device and/or electrical stimulator and/or electrodes can be configured to provide a voltage that is less than about 100 V/cm. In some embodiments, the electrical stimulator can be configured to apply an electrical pulse of less than about 500 V/cm. Other configurations providing for other voltages and/or voltage ranges are also possible. In some embodiments, any one of more of the voltages can be applied over 0.1 cm or more, and up to and include 20 cm.

In some embodiments, the electrical stimulator includes at least a first electrode (shown as the balloon 6 in FIG. 1) configured to contact a tissue near the ultrasound head when the device is in use on an endoscope. In some embodiments, the electrical stimulator includes additional electrodes configured to contact a tissue near the ultrasound head. For example, in some embodiments, the electrical stimulator includes two, three, four, five, or more than five electrodes. In some embodiments, electrical stimulation can occur between two or more electrodes. In some embodiments, the electrodes are arranged in an array, so as to provide an array based system for electrical stimulation. While depicted as a balloon in FIG. 1, any type and/or arrangement of electrodes can be used in various embodiments provided herein, as long as electrical stimulation can be provided to a region of interest. In some embodiments, this can include an external ultrasound device, system, and/or method, which can be applied in conjunction with a separate electrical stimulator. Thus, an endoscopic stimulator need not be used with the electrical stimulator in all embodiments.

In some embodiments, at least the first electrode includes an electrically conductive elastomer. In some embodiments, at least the first electrode includes a doped or modified silicone or fluorosilicone rubber and/or other electrically conductive elastomer. For example, the first electrode can include a doped or modified polybutadiene, chloroprene, butyl rubber, styerene butadiene, nitrile rubber, EPM, ECO, polyacrylic rubber, FKM, FEPM, FFKM, polyether block amide, CSM, or EVA. Other materials, such as other doped or modified elastomers, are also possible. In some embodiments, any material suitable for use as an electrode can be employed.

As will be appreciated by one of skill in the art, the electrodes can take on a variety of shapes and/or configurations and be placed in a variety of locations. In some embodiments, the electrode is integrated into part of the probe. In some embodiments, the electrode is associated with the probe, but can be separate from the probe, for example, a separate cable that is associated with the external surface of the US probe. In some embodiments, the electrode can simply be an exposed conducting surface on the US probe. In some embodiments, the electrode can be separate from the probe. In some embodiments, the electrode can be a specific part and/or structure of the US probe.

As shown in FIG. 1, in some embodiments, the electrode includes and/or is part of a balloon. The balloon can include an electrically conductive elastomer, such as those described above. In some embodiments, the electrode includes more than one balloon. In some embodiments, the balloon further includes an electrically insulating elastomer section and/or layer. In some embodiments, the electrically insulating elastomer is positioned inside the electrically conductive elastomer. In some embodiments, the ultrasound head is positioned internal to the electrically conductive elastomer. In some embodiments, the ultrasound head is positioned internal to the electrically insulating elastomer. Positioning the ultrasound head internal to the electrically insulating elastomer can shield the piezo transducer of the ultrasound. In some embodiments, no balloon is provided and/or the electrode is separate from the balloon arrangement.

In some embodiments, the ultrasound head is positioned internal to the electrically conductive elastomer (such as when the electrode is, or is part of, the balloon), which can allow matching of the acoustic connection of the ultrasound transducer to the electrical connection of the electrode. This can cause the electrode field and the ultrasound transducer field to overlap, which can help ensure that the electrical stimulation and acoustic stimulations match and can be synchronized. This is not required for all embodiments.

In some embodiments, the electrical stimulator includes a monopolar electrode arrangement. In some embodiments, the patient can be attached to a return electrode which is connected to the stimulator. The return electrode can be any type of electrode and can include, for example, a large metal plate or flexible metalized plastic pad. The electric current can flow from the active electrode, through the body to the return electrode, and then back to the stimulator.

In some embodiments, the electrical stimulator includes a dipolar electrode arrangement. The return electrode can be in close proximity with the active electrode. In some embodiments, the return electrode is a probe which can be inserted into the tissue. For example, a needle (e.g., a EUS-FNA needle) can serve as the return electrode.

In some embodiments, the ultrasound head and the electrical stimulator are on the same probe. This can aid in ensuring the fields of the ultrasound head and the electrical stimulator overlap, as described above.

In some embodiments, the device for ultrasound imaging further includes a computer configured to receive a signal from the ultrasound head. In some embodiments, the device for ultrasound imaging further includes a timing system configured to coordinate an ultrasound signal with an excitation signal from the electrical stimulator. In some embodiments, one or more of these parts can be set up and/or relocated in another section of a system.

In some embodiments, a system for electric stimulation enhanced ultrasound imaging is provided. The system can include an ultrasound unit, an electrical stimulator, and a controller configured to apply an electric pulse of less than about 100 V/cm through the electrical stimulator. In some embodiments, the ultrasound unit can include an ultrasound transducer. The ultrasound transducer and the electrical stimulator can be located next to one another.

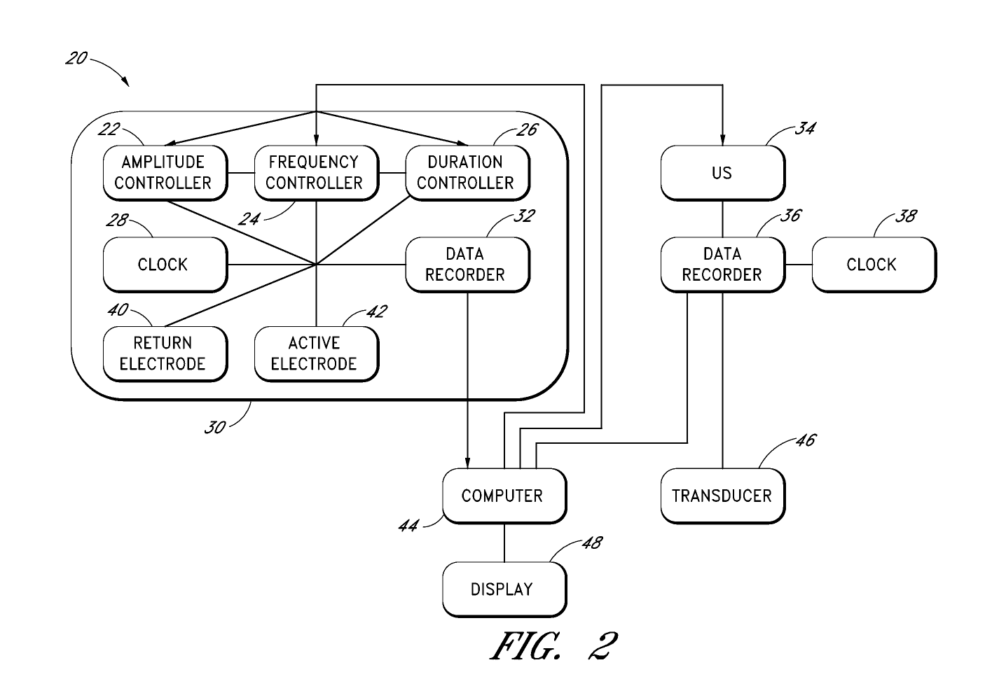

FIG. 2 is a drawing depicting some embodiments of a system 20 for electric stimulation enhanced ultrasound imaging. The system can include an electrical stimulator 30 that include an amplitude controller 22, a frequency controller 24, a duration controller 26, a clock 28, and a data recorder 32. The amplitude controller can be configured to control the amplitude of the electrical stimulation applied. The frequency controller 24 can be configured to control a frequency of the electrical stimulation applied. The duration controller 26 can be configured to control a duration of the applied electrical stimulation. The clock 28 can be configured to record a time that electrical stimulation is applied. The data recorder 32 can be configured to record characteristics of an electrical stimulation applied and/or a time an electrical stimulation is applied. Any of a variety of instruments can be used as the controllers, clocks, and/or recorders. These aspects can be included in the computer that controllers the ultrasound and/or electrical stimulator. In some embodiments, one or more of these components is provided outside of the electrical stimulator, e.g., by the computer etc.

The stimulator 30 can also include and/or provide a connection with a return electrode 40 and an active electrode 44. As described herein, these can be arranged in a monopolar configuration, a dipolar configuration, and/or other configurations. In some embodiments, these components can be combined and/or in electrical communication with one another as part of the electrical stimulation component. In some embodiments, the stimulator 30 need not include all of the structures shown in FIG. 2, and can instead simply be an active electrode and optionally one or more of the other components.

The system 20 also includes an ultrasound unit 34. The ultrasound unit 34 can be in communication with a data recorder 36 and a clock 38. The clock 38 can be configured to record a time at which an ultrasound image is taken. The data recorder 36 can be configured to record ultrasound images and/or times at which ultrasound image is taken. Both the stimulator 30 and the ultrasound unit 34 can be connected and/or connectable to a computer 44. The computer 44 can be configured to synchronize electrical stimulation with ultrasound imaging. The computer 44 can be connected to a display 48 which can be configured to display a number of items including ultrasound images, display changes in ultrasound images caused by electrical stimulation, display stimulation applied, display times at which stimulations were applied, etc. Any suitable type of ultrasound unit and/or ultrasound head can be employed.

In some embodiments, the controller is configured to apply an electrical pulse of less than about 10 V/cm. In some embodiments, the controller can be configured to apply an electrical pulse of less than about 50 V/cm. In some embodiments, the controller can be configured to apply an electrical pulse of less than about 150 V/cm. In some embodiments, the controller can be configured to apply an electrical pulse of less than about 200 V/cm. In some embodiments, the controller can be configured to apply an electrical pulse of less than about 500 V/cm. In some embodiments, the controller is configured to apply an electrical pulse in a square wave form. In other embodiments, the waveform of the pulse need not be square and can be, for example, curved and/or ramped.

In some embodiments, the electrical stimulator includes an electrically conductive elastomer, such as those described herein. In some embodiments, the electrical stimulator includes a balloon. The balloon can include an electrically conductive elastomer, such as those described above.

In some embodiments, the electrical stimulator includes an active electrode in electrical communication with a return electrode. Any electrical stimulation system can be employed in the system, including those provided herein.

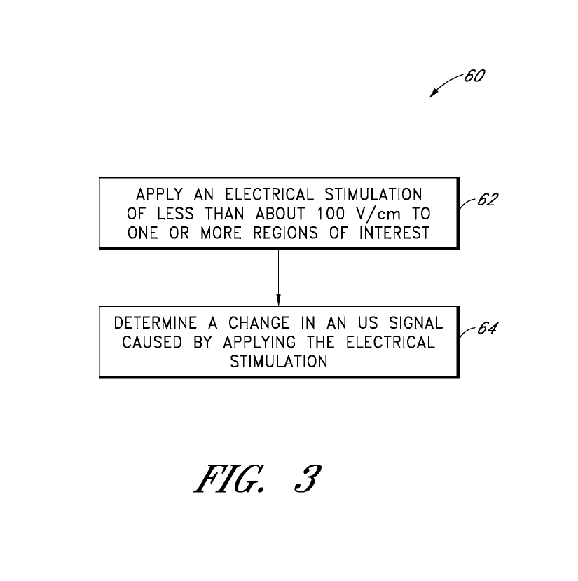

As shown in FIG. 3, a method 60 for electric stimulation enhanced ultrasound imaging is provided. The method can include step 62 which involves applying an electrical stimulation of less than about 100 V/cm to one or more regions of interest. The method 60 may also includes step 64 which involves determining a change in an ultrasound signal caused by applying the electrical stimulation.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Any change in the ultrasound signal due to the electrical stimulation can be used for various applications. For example, determining a change in the ultrasound signal can include determining an ultrasound signal before electrical stimulation and determining an ultrasound signal after electrical stimulation. In some embodiments, determining the change in the ultrasound signal includes determining an ultrasound signal before electrical stimulation and determining an ultrasound signal during electrical stimulation. In some embodiments, determining the change in the ultrasound signal includes determining an ultrasound signal during electrical stimulation and determining an ultrasound signal after electrical stimulation. These signals can then be compared to determine the differences between them, with these differences providing additional information regarding the regions of interest.

In some embodiments, determining the ultrasound signal is an ongoing process, and can occur throughout and/or during one or more stimulations. In some embodiments, one can determine a signal by providing a first ultrasound reading of a region of interest during a first set of conditions of electrical stimulation, and continue to monitor the ultrasound signal as one provides an electrical stimulation. In other words, an ultrasound reading can occur before, during, or after the electrical stimulation. In some embodiments, when an ultrasound is being determined during the electrical stimulation, it may not be required to compare a first and second ultrasound image, as the change of the ultrasound image can be apparent from a single (for example continuously updated) ultrasound image (which can be, for example, viewed in real time).

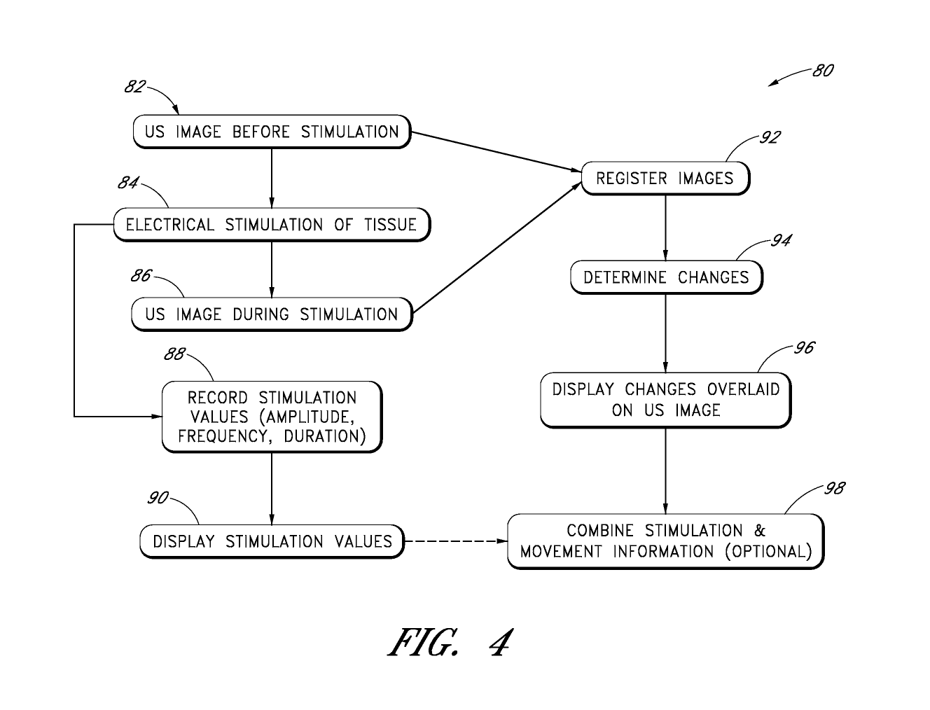

FIG. 4 depicts some embodiments of methods for electric stimulation enhanced ultrasound imaging 80. The method can include determining a change in an ultrasound signal (such as an ultrasound image) by determining an ultrasound signal before electrical stimulation and determining an ultrasound signal during and/or after electrical stimulation. The method can include the process of obtaining an ultrasound image before stimulation 82 and, optionally, the process of registering the image 92. The method can include the process of electrically stimulating tissue 84, the process of obtaining an ultrasound image during stimulation 86, the process of registering the ultrasound image obtained during stimulation 92, and the process of recording stimulation values or characteristics 88. The method 80 can also include the process of displaying the stimulation values 90. The method can also include the process of determining changes between the ultrasound images 94. The method can further include the process of displaying the changes overlaid on an ultrasound image 96. The method can also optionally include the process of combining stimulation and change information 98. In some embodiments, one or more of these processes or the results thereof can be displayed to a user on a display screen and/or stored on a computer readable medium.

In some embodiments, applying an electrical stimulation includes applying at least a pulse of electricity in an area or subpart of an area undergoing ultrasound imaging. In some embodiments, all or substantially all of the area being observed by ultrasound is stimulated with the pulse of electricity. In some embodiments, a subpart of the area is stimulated by electricity (for example 50% of the area of an ultrasound image could be subjected to an electrical pulse having a prescribed intensity). In some embodiments, 10% or more of the area of the ultrasound image can be subject to the electrical pulse. In some embodiments, one can arrange the system and/or application of the pulse such that one has control regions, for example to determine, in real time, whether the region of interest is responding compared to a similar tissue which can be adjacent, and is not stimulated by the electrical stimulation, but still being measured by the ultrasound. It is not necessary that electrical stimulation be applied in all instances of ultrasound imaging. Imaging may occur without electrical stimulation.

In some embodiments more than one pulse can be applied between and/or during the ultrasound interrogations. In some embodiments, applying the electrical stimulation includes applying a series or “train” of electrical pulses in an area undergoing ultrasound imaging. In some embodiments, the pulses of the train can be similar and/or identical (for example, in voltage, duration, frequency, etc.). In some embodiments, one or more of the pulses can differ. For example, applying the electrical stimulation can include applying at least two electrical pulses, wherein the at least two electrical pulses differ by at least one of frequency, amplitude, or duration.

In some embodiments, the duration of stimulation ranges from about 0.1 ms to about 3 s. In some embodiments, the duration of stimulation ranges from about 1 ms to about 3 s. In some embodiments, the duration of stimulation ranges from about 1 ms to about 1 s. In some embodiments, the duration of stimulation ranges from about 1 s to about 2 s. In some embodiments, the duration of stimulation ranges from about 2 s to about 5 s. Other durations are also possible and are not limiting.

In some embodiments, applying the electrical stimulation includes applying about 0.1 V/cm to about 10 V/cm. In some embodiments, applying the electrical stimulation includes applying about 0.1 V/cm to about 5 V/cm. In some embodiments, applying the electrical stimulation includes applying about 5 V/cm to about 10 V/cm. Other voltage ranges, including those above 10 V/cm, are also possible. In some embodiments, the voltage applied is less than that required to destroy and/or damage cells. In some embodiments, the voltage applied does not destroy and/or damage cells.

In some embodiments, applying the electrical stimulation includes an electrical stimulation of about 0.01 Hz to about 100 Hz. In some embodiments, applying the electrical stimulation includes an electrical stimulation of about 100 Hz to about 500 Hz. In some embodiments, applying the electrical stimulation includes an electrical stimulation of about 500 Hz to about 1 kHz. In some embodiments, applying the electrical stimulation includes an electrical stimulation of about 1 kHz to about 10 kHz. In some embodiments, applying the electrical stimulation includes an electrical stimulation of about 10 kHz to about 100 kHz. In some embodiments, the stimulation is in the sub-Hertz range. In some embodiments, stimulations can take 10 seconds or more. Other frequencies are also possible.

In some embodiments, a method of electric stimulation enhanced ultrasound imaging includes determining and/or recording one or more of 1) a value of electrical stimulation applied that causes a change in the ultrasound signal, 2) the change in the ultrasound signal, and 3) a combination of the value of electrical stimulation applied that caused the change in the ultrasound signal and the change in the ultrasound signal. In some embodiments, an initial value of electrical stimulation applied is below the value required to cause a change in the ultrasound signal (for example, a “threshold value”). In some embodiments, pulses of increasing voltage, frequency, and/or duration can be applied until a change in the ultrasound signal is observed. The value of electrical stimulation can be increased by varying one or more of frequency, amplitude, and/or duration.

In some embodiments, a method of electrical stimulation enhanced ultrasound imaging includes comparing one or more of 1) a value of electrical stimulation applied that caused a change in the ultrasound signal, 2) a change in the ultrasound signal, and 3) a combination of the value of electrical stimulation applied that caused the change in the ultrasound signal and the change in the ultrasound signal, with one or more a pre-existing, similar, database results derived from one or more positive and/or negative controls for a tissue sample having known properties (for example having a tumor, not having a tumor, tissue type, tissue condition, tissue age, etc.). In some embodiments, comparison to a pre-existing database can aid in determining whether the data is consistent with a particular tissue type or disease state (for example, presence or absence of a tumor). In some embodiments, the value of electrical stimulation applied to cause a change in the ultrasound signal can be used to create such a database by correlating such values (such as voltage step, duration, frequency, etc) and the resulting changes in the tissue being stimulated with the properties of a known tissue and/or region of interest (for example, tumor in the pancreas).

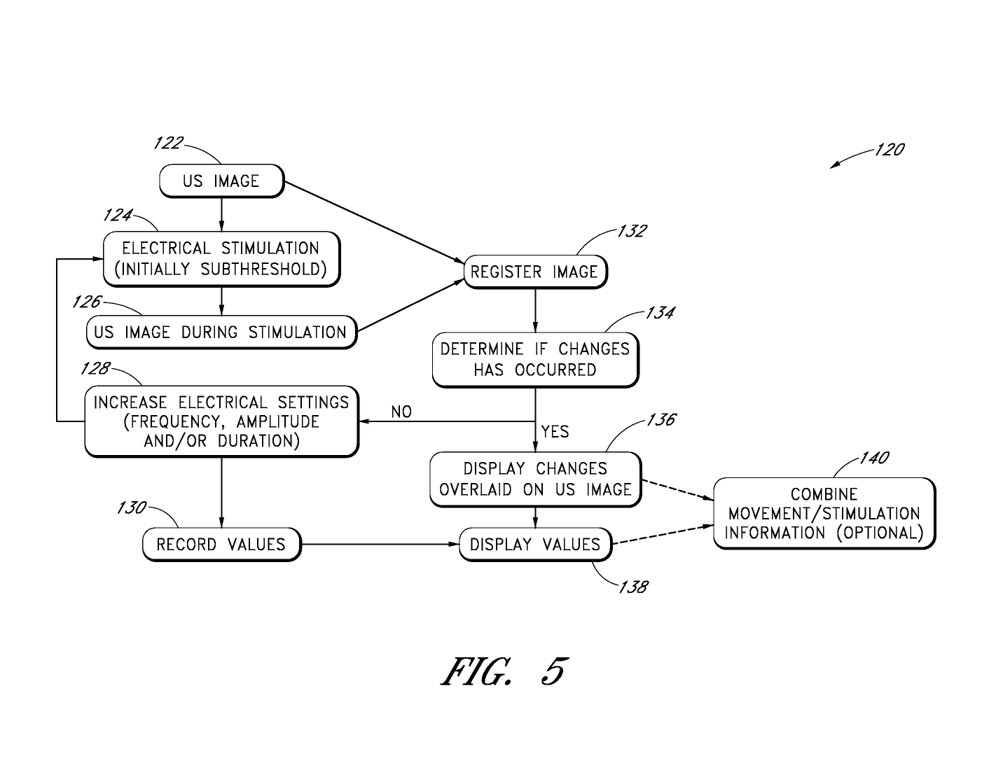

FIG. 5 depicts some embodiments of a method 120 in which electrical stimulation values can be increased until a change in ultrasound signal is measured. The method 120 includes the process of obtaining an ultrasound image 122 and the process of registering the image 132. The method 120 includes the process of electrically stimulating the tissue at a sub-threshold value 124. A sub-threshold value is one that will result in no change or very minimal change in ultrasound value. Process 126 includes obtaining an ultrasound image during stimulation (which can optionally be adjusted to obtaining the signal after stimulation). The ultrasound image is registered in process 132. Process 134 includes determining whether a change has occurred in the ultrasound signal based on the image from before and during (and/or after) electrical stimulation. If no change has occurred, the electrical stimulation value is increased in process 128 by increasing one or more of frequency, amplitude, or duration. Processes involving electrical stimulation (at the higher level) 124, obtaining an ultrasound image during stimulation 126, registering the image 132, and determining whether a change has occurred 134 are then repeated. If, in process 134, it is determined that a non-zero change has occurred, the changes are displayed by being overlaid on the ultrasound image in process 136. The method 120 also includes the process 130 of recording the different values of electrical stimulation applied. In process 138, the electrical stimulation values and/or the ultrasound images/changes can be displayed. The method 120 can also include the process of combining the change or movement and stimulation information 140. Using this repeatable process, one can not only test a tissue sample for properties that reveal its sensitivity to electrical stimulation (observable via ultrasound), but also establish a database of ultrasound observable electrical stimulation properties by performing the method on tissues with known properties and/or characteristics. Such databases can be created for any tissue that can be subjected to ultrasound and some form of electrical stimulation. In some embodiments, the database (and/or device) can be configured for use in the gastrointestinal, pulmonary systems, adjacent organs (e.g. pancreas, liver, lung, prostate, etc), and/or associated vasculature and lymphatic systems. Thus, methods for testing such areas are also contemplated. In some embodiments, the system is implemented externally and can be used to interrogate musculature. In some embodiments, miniaturized embodiments can be used for neurological interrogations.

In some embodiments, the one or more regions of interest includes one or more of at least a part of an organ, at least a part of a membrane, at least a part of a vasculature, at least a part of a tumor inside an organ, at least a part of a tumor inside a vasculature, and at least a part of a tumor inside a membrane. Other regions are also possible. For example, in some embodiments, the one or more regions of interest includes a foreign element within one of the above described regions. The foreign element can be, for example, a contrasting agent and/or a controlled release drug system, which can be monitored and released in controlled fashion using the current invention. In some embodiments, two, three, four, five, or more regions of interest can be electrically stimulated and imaged.

In some embodiments, applying an electrical stimulation and determining a change in an ultrasound signal caused by applying the electrical stimulation are cyclically repeated. In some embodiments, at least one of an initial value of electrical stimulation to be applied, a final value of electrical stimulation to be applied, and a time between subsequent applications of electrical stimulation is pre-programmed into a computer that controls applying the electrical stimulation. Each subsequent application of electrical stimulation can be programmed to increase (and/or decrease) by a predetermined amount. The subsequent applications of electrical stimulation can be programmed to increase (and/or decrease) linearly or non-linearly. In some embodiments, the tissue can be electrically stimulated in a train, such that the cumulative effects of the train of electrical pulses provides a unique test condition for the tissue being stimulated. In other embodiments, while more than one pulse can be applied to the same region of interest, the tissue is allowed a sufficient period of time to recover between pulses, such that the impact of the first pulse on the tissue is removed or minimized before the impact of the second pulse, thereby allowing repeated sampling of the same region of interest under the same electrical conditions (rather than providing a train as the electrical condition).

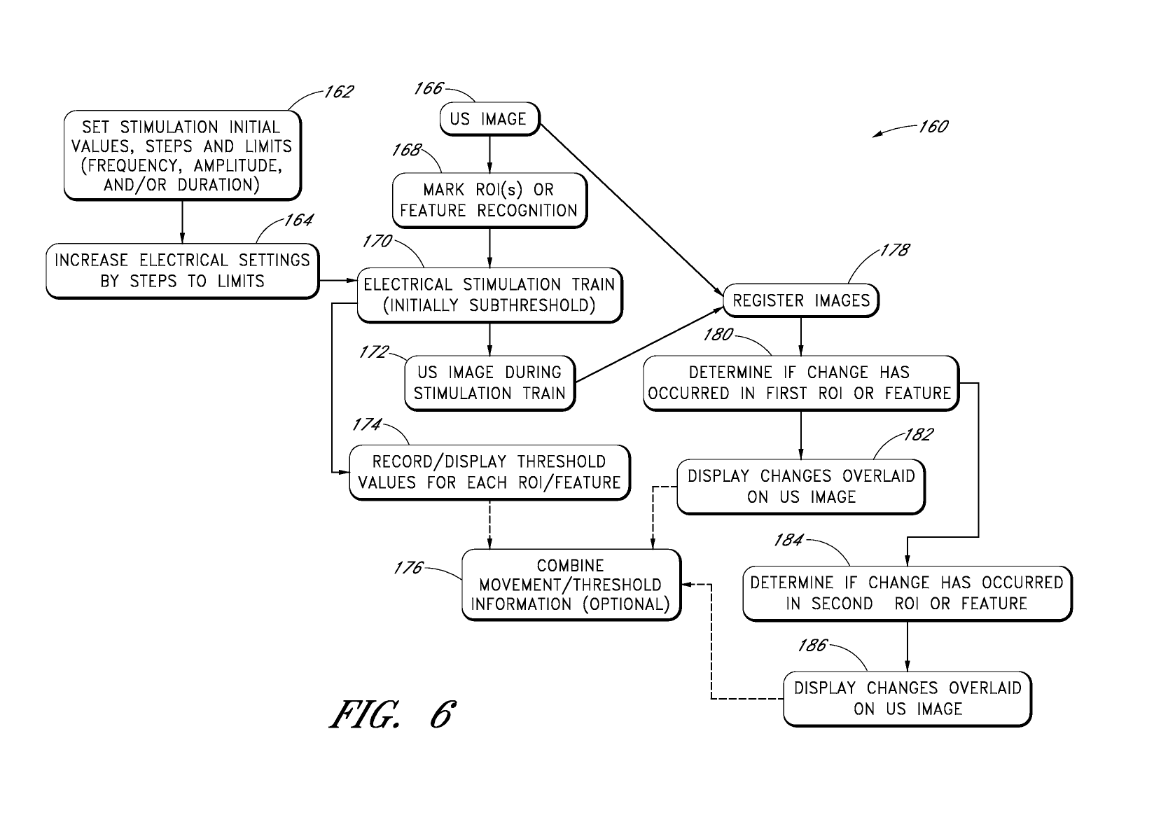

FIG. 6 depicts some embodiments of a method 160 in which multiple regions of interest are stimulated using cyclical repetition of electrical stimulation and determining a change in an ultrasound signal caused by the electrical stimulation. The method 160 includes obtaining ultrasound images of the regions 166, marking the region or feature 168, and, optionally, registering the images 178. Process 162 includes setting up an electrical stimulation train, including at least one of setting initial electrical stimulation values, steps between values, and limits to the values. The steps can vary the frequency, amplitude, and/or duration of the values. Process 170 includes applying the electrical stimulation train to the region of interest. Each stimulation includes the process of obtaining an ultrasound image 172 during (or quickly after) the stimulation. The image is registered in process 178. Process 180 includes determining whether a change in ultrasound signal has occurred for the first region of interest or first feature. Any changes can be displayed (for example overlaid) on the ultrasound image in process 182 (any of the ultrasound images can be used, but displaying it against the resting state can be a standardized approach if desired). Process 184 includes determining whether a change in ultrasound signal has occurred for a second region of interest or second feature. One or more of the changes can be displayed overlaid on the ultrasound image in process 186. One can then record and/or display the threshold (or change) values for each region of interest or feature 182. The change or movement and threshold information can be combined as shown in process 176. If more than two regions of interest or features are being stimulated or examined, additional processes including determining whether a change has occurred and displaying changes can be added after the process of registering the images 178. Combining data from more than one region of interest can determine relative changes in these different regions, and can be used for diagnostic indication (for example, by comparing to a database). In some embodiments, the data can simply be taken from two different areas of the same ultrasound scan. In some embodiments, the regions of interest can be far enough apart that two separate ultrasounds are performed and then compared.

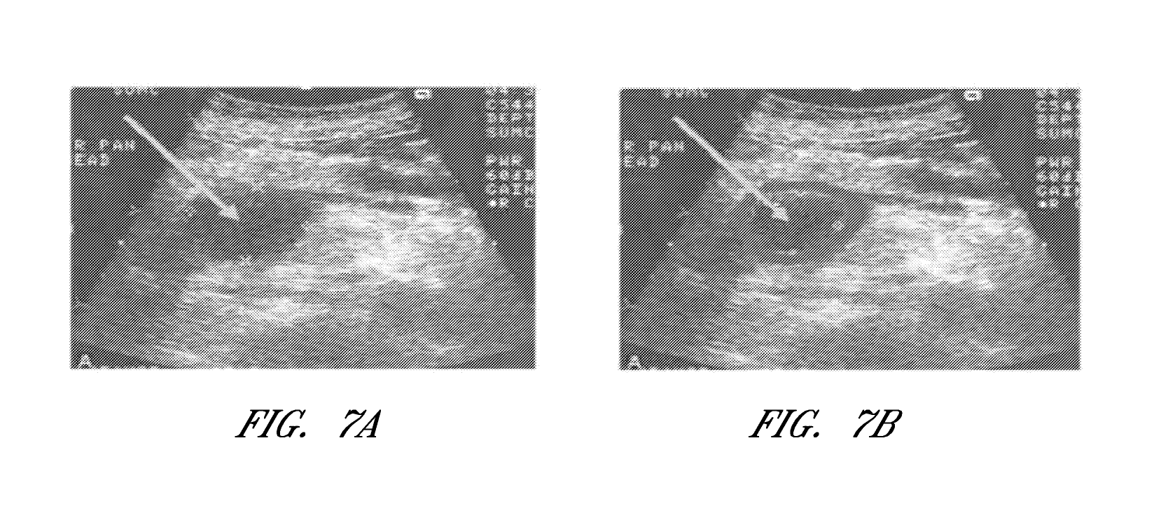

FIG. 7A depicts a hypothetical ultrasound image of a region of interest. FIG. 7B depicts an ultrasound image displaying an example of a hypothetical change and how such a change can be indicated (for example, the circled area) caused by electrical stimulation. In some embodiments, determining a change in the ultrasound signal is performed by a computer. In some embodiments, determining a change in the ultrasound signal is performed manually, for example, by looking at two different ultrasound images.

In some embodiments, a method for electrical stimulation and ultrasound imaging is provided. The method includes (a) acquiring an ultrasound image of one or more regions of interest, (b) applying a pulse of electrical stimulation to the one or more regions of interest, and (c) acquiring an ultrasound image of the one or more regions of interest during application of the pulse of electrical stimulation.

In some embodiments, the polarity of the electrical field applied during the electrical stimulation can be reversed during the process. In some embodiments, values below those causing a non-zero change can be employed, for example, those in the μV/cm to mV/cm range. Cyclical electrical stimulations can be applied at such values (as well as higher values). In some embodiments, pauses are provided between subsequent stimulation applications. As noted above, some pauses can allow the tissue to rest and return to normal before a subsequent stimulation. Such pauses are generally longer than the pauses in train stimulation, and can be denoted as “recovery pauses”. In some embodiments, ultrasound measurements are taken continuously. As noted above, in some embodiments, after reaching a change in ultrasound signal, the system can alternate between stimulating tissue to cause a change and resting (all the way to a fully recovered state), several times, in order to improve the quality of the data obtained.

In some embodiments a change in ultrasound signal can be observed as mechanical strain, movement, and/or other changes in the observed signal.

In some embodiments, electrical stimulating enhanced ultrasound can aid in improving the resolution of endoscopic ultrasound images in general. In some embodiments, electrical stimulating enhanced ultrasound can aid in improving the resolution of endoscopic ultrasound images which can improve the ability to diagnose and stage tumors. Due to structural and functional differences, tumor tissue can have different conductive and permittive properties. These different properties can be exploited by electrical stimulating enhanced ultrasound for identifying boundaries and for determining the penetration through membranes into underlying structures which can be integral in current staging methodologies. Furthermore, statistical analysis of the threshold values for electrical stimulation to cause a change in ultrasound signal in a particular region of interest can afford diagnostic capabilities complementary with current methodologies (e.g., such as in endoscopic ultrasound and FNA biopsy methodologies). In some embodiments, the electrical stimulating enhanced ultrasound can improve imaging capability while providing minimal tissue disruption.

In some embodiments, the system can incorporate a return electrode pad or an electrode integrated into an FNA needle. In some embodiments, the method can be embodied in a software system that runs algorithms for the integration of the system (e.g., the enhanced display of ultrasound information). In some embodiments, the software can statistically analyze the data (e.g., comparing the data to a pre-existing database). In some embodiments, cloud services can be used to offer the technology described herein as a diagnostic service.

Combined electric field and ultrasound (CEFUS) has been described for therapeutic applications, such as anti-tumor treatment. In contrast, some of the present embodiments avoid Joule heating and/or electroporation effects. In some embodiments, the method and/or device is configured to avoid damage to the tissue and can avoid employing exponential or square wave electric pulses with a peak of 1000 V/cm for 1 ms×2 at 1 Hz. In some embodiments, the method and/or device is configured to avoid and/or minimize a thermal effect, while providing a non-thermal effect. Thermal effects are due to Joule heating in tissue, whereas the non-thermal effects can be due to electrokinetic effects, piezoelectric effects, electrorestriction, and electrophoresis. Thus, in some embodiments, the methods and/or devices can be configured so as to provide at least one of an electrokinetic effect, piezoelectric effect, electrorestriction, and/or electrophoresis, without providing a significant Joule heating effect, such as damaging and/or destroying cells via heat.

The electrokinetic phenomenon can include several options, including electrophoresis, electroosmosis, sedimentation potential, and/or streaming potential. The electrokinetic effects of a colloidal in an electrolyte solution have well established parameters: the applied electric field, the Zeta potential, and the surface electrical properties of the particle. Without intending to be limited by theory, in some embodiments, the effects of a low electric field strength, on the order of a few V/cm, on tissue have been demonstrated to be due to the electrokinetic effect, and not the other non-thermal effects. In some embodiments, the amount and/or characteristics of the applied electrical stimulation can be sufficient to provide an electrokinetic effect, without, or with minimal cell damage due to any heating effects. In some embodiments, the stimulation is sufficient to induce an inverse piezoelectric effect, which occurs in tissue when certain materials, such as collagen fibers, are oscillated in an alternating electric field. This causes an acoustic effect. This has been proposed as a system to measure the acoustic effects of a stimulated tissue using ultrasound or with RF stimulation.

In an illustrative embodiment, any of the operations, processes, etc. described herein can be implemented as computer-readable instructions stored on a computer-readable medium. The computer-readable instructions can be executed by a processor of a mobile unit, a network element, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

### EXAMPLES

### Example 1

### Determining a Voltage Threshold

A first ultrasound image is obtained of a region of the pancreas suspected of containing a tumor. A sub-threshold voltage stimulation of 0.1 V/cm is applied to the region. A second ultrasound image is obtained during the stimulation. A computer is used to compare the first and second ultrasound images and determines that no difference is observed. The region is allowed 3 seconds of rest and is then stimulated again using a voltage stimulation that is 0.1 V/cm greater than the previous value. A third ultrasound image is obtained during the second stimulation. A computer is used to compare the first and third images and determines whether a change has occurred. The cycle of applying an electrical stimulation of increasing amplitude and obtaining an ultrasound image during stimulation is repeated until the computer determines a change has occurred. The stimulation values and an ultrasound image displaying the change are displayed. This allows one to determine a voltage threshold for testing other areas of the same tissue for possible tumors.

### Example 2

### Detecting a Lesion

A first ultrasound image is obtained of a prostate gland that is suspected of having a lesion. A train of 10 electrical pulses, of 10 V/cm, each pulse lasting 100 ms is applied to the tissue. An ultrasound image is obtained at the end of each of the 10 pulses. After the train of electrical stimulation is completed, the ultrasound images, times of application, and electrical stimulation values are displayed. While the prostate gland will change during the electrical stimulation, the tissue around a lesion will change differently, and thus be clearer by contrasting the ultrasound taken during the train to the ultrasound taken before the train. The difference can be stronger in the second half of the series of 10 pulses, and thus, the comparison can be made using that information in contrast to the first ultrasound. Alternatively, the ultrasound data from the train of 10 can be combined to provide a lower amount of noise, and the averaged image can be compared to the first ultrasound (the time between each pulse will then have to be extended so that the tissue is allowed to recover fully between the pulses).

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A device for ultrasound imaging, the device comprising:
an ultrasound head; and
an electrical stimulator positioned proximal to the ultrasound head, wherein the electrical stimulator is configured to apply an electrical pulse of less than about 100 V/cm.

2. The device of claim 1, wherein the electrical stimulator comprises at least a first electrode configured to contact a tissue near the ultrasound head, when the device is in use on an endoscope.

3. The device of claim 2, wherein the first electrode comprises an electrically conductive elastomer.

4. The device of claim 3, wherein the electrically conductive elastomer comprises at least one of a doped silicone or a fluorosilicone rubber.

5. The device of claim 2, wherein the electrode comprises a balloon.

6. The device of claim 5, wherein the electrode comprises an electrically conductive elastomer.

7. The device of claim 6, wherein the balloon further comprises an electrically insulating elastomer.

8. The device of claim 7, wherein the electrically insulating elastomer is positioned inside the electrically conductive elastomer.

9. The device of claim 8, wherein the ultrasound head is positioned internal to the electrically conductive elastomer.

10. The device of claim 1, wherein the electrical stimulator comprises a monopolar arrangement.

11. (canceled)

12. The device of claim 1, wherein the ultrasound head and the electrical stimulator are on a same probe.

13. The device of claim 1, further comprising a computer configured to receive a signal from the ultrasound head.

14. The device of claim 1, further comprising a timing system configured to coordinate 1) an ultrasound signal with, 2) an excitation signal from the electrical stimulator.

15. A system for electric stimulation enhanced ultrasound imaging, the system comprising:
an ultrasound unit;
an electrical stimulator; and
a controller, configured to apply an electric pulse of less than about 100 V/cm through the electrical stimulator.

16. The system of claim 15, further comprising a data acquisition unit in communication with the ultrasound unit, wherein the data acquisition unit is configured to record ultrasound images received from the ultrasound unit and record a time of imaging of the ultrasound images.

17. The system of claim 15, wherein the ultrasound unit comprises an ultrasound transducer.

18. The system of claim 17, wherein the ultrasound transducer and the electrical stimulator are located next to one another.

19. (canceled)

20. (canceled)

21. The system of claim 15, wherein the electrical stimulator comprises a balloon.

22. The system of claim 21, wherein the balloon comprises an electrically conductive elastomer.

23. (canceled)

24. (canceled)

25. (canceled)

26. A method for electric stimulation enhanced ultrasound imaging, the method comprising:
applying an electrical stimulation of less than about 100 V/cm to one or more regions of interest; and
determining a change in an ultrasound signal caused by applying the electrical stimulation.

27. The method of claim 26, wherein determining the change in the ultrasound signal comprises determining an ultrasound signal before electrical stimulation and determining an ultrasound signal after electrical stimulation.

28. (canceled)

29. (canceled)

30. The method of claim 26, wherein applying the electrical stimulation comprises applying at least one pulse of electricity in an area undergoing ultrasound imaging.

31. The method of claim 30, wherein applying the electrical stimulation comprises applying a train of electrical pulses.

32. (canceled)

33. (canceled)

34. (canceled)

35. (canceled)

36. The method of claim 26, wherein determining the change in the ultrasound signal is performed on a computer.

37. (canceled)

38. The method of claim 26, wherein at least one of an initial value of electrical stimulation to be applied, a final value of electrical stimulation to be applied, and a time between subsequent applications of electrical stimulation is pre-programmed into a computer that controls applying the electrical stimulation.

39. The method of claim 26, further comprising recording one or more of 1) a value of electrical stimulation applied that caused a non-zero change in the ultrasound signal, 2) the non-zero change in the ultrasound signal, and 3) a combination of the value of electrical stimulation applied that caused the non-zero change in the ultrasound signal and the non-zero change in the ultrasound signal.

40. The method of claim 39, further comprising comparing one or more of 1) the value of electrical stimulation applied that caused a non-zero change in the ultrasound signal, 2) the non-zero change in the ultrasound signal, and 3) the combination of the value of electrical stimulation applied that caused the non-zero change in the ultrasound signal and the non-zero change in the ultrasound signal, with a pre-existing database results derived from one or more positive controls for a known tissue sample.

41. A method for electrical stimulation and ultrasound imaging, the method comprising:
(a) acquiring an ultrasound image of one or more regions of interest;
(b) applying a pulse of electrical stimulation to the one or more regions of interest; and
(c) acquiring an ultrasound image of the one or more regions of interest during application of the pulse of electrical stimulation.
